(19) United States
(12) Patent Application Publication
(10) Pub. No.: US 2022/0249317 A1
(43) Pub. Date: Aug. 11, 2022

(54) Hair Treatment Cap Apparatus

(71) Applicants: Arinola Okinbaloye, Madison, AL (US); Babatunde A. Obembe, Madison, AL (US)

(72) Inventors: Arinola Okinbaloye, Madison, AL (US); Babatunde A. Obembe, Madison, AL (US)

(21) Appl. No.: 17/171,508

(22) Filed: Feb. 9, 2021

(51) Int. Cl. A61H 7/00; A45D 19/14; A61M 35/00

(52) U.S. Cl. CPC A61H 7/006; A45D 19/14; A61H 7/004; A61M 35/10; A61H 2201/1607; A61H 2201/105; A61H 2205/021; A61H 2201/1692; A61H 2201/1207; A61H 2201/5025; A61H 2201/5043; A61H 2201/1645; A61H 2201/165

(57) ABSTRACT

A hair treatment cap apparatus for product application and scalp massage for hair loss prevention includes a cap body having an inner layer and an outer layer joined at a cap edge and defining a cap bladder. The inner layer has a plurality of release apertures extending therethrough and the outer layer has a fill aperture extending therethrough to the cap bladder. The cap bladder holds a liquid hair treatment that is filled through the fill aperture and dispersed through the plurality of release apertures. The cap body is sufficiently flexible to conform to the shape of a wearer's head. The cap edge is elasticized to form a waterproof barrier around the user's head. A plurality of massage motors is coupled to the cap body to vibrate a plurality of nubs coupled to the inner layer. A controller coupled to the cap body operates the plurality of massage motors.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

The disclosure relates to hair treatment devices and more particularly pertains to a new hair treatment device for product application and scalp massage for hair loss prevention. The present device includes a plurality of rubber nubs activated by a plurality of vibrating motors for scalp stimulation. There is also a membrane with a plurality of release apertures to evenly distribute liquid treatments.

### (2) Description of Related Art Including Information Disclosed Under 37 CFR 1.97 and 1.98

The prior art relates to hair treatment devices. Known devices include scalp massagers that employ rigid caps and a single vibrational device. These devices lack a flexible and comfortable cap that is also waterproof, as well as a plurality of vibrating motors.

### BRIEF SUMMARY OF THE INVENTION

An embodiment of the disclosure meets the needs presented above by generally comprising a cap body having an inner layer and an outer layer joined at a cap edge and defining a cap bladder. The inner layer has a plurality of release apertures extending through to the cap bladder. The outer layer has a fill aperture extending through to the cap bladder. The cap bladder is configured to hold a liquid hair treatment that is filled through the fill aperture and dispersed through the plurality of release apertures. The cap body is sufficiently flexible to conform to the shape of a wearer's head. The cap edge is elasticized to form a waterproof barrier around the user's head. A plug is coupled to the cap body to selectively engage the fill aperture. A plurality of nubs is coupled to the inner layer of the cap body. A plurality of massage motors is coupled to the cap body. The plurality of massage motors causes each of the plurality of nubs to vibrate. A controller is coupled to the cap body and is in operational communication with the plurality of massage motors. A battery is coupled within the controller and is in operational communication with the controller.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is an isometric view of a hair treatment cap apparatus according to an embodiment of the disclosure.

FIG. 2 is a front in-use view of an embodiment of the disclosure.

FIG. 3A is a rear elevation view of an embodiment of the disclosure.

FIG. 3B is a rear elevation view of an embodiment of the disclosure.

FIG. 4 is a bottom plan view of an embodiment of the disclosure.

FIG. 5 is a cross-sectional view of an embodiment of the disclosure along the line 5-5 of FIG. 4.

embodiment of the disclosure.

### DETAILED DESCRIPTION OF THE INVENTION

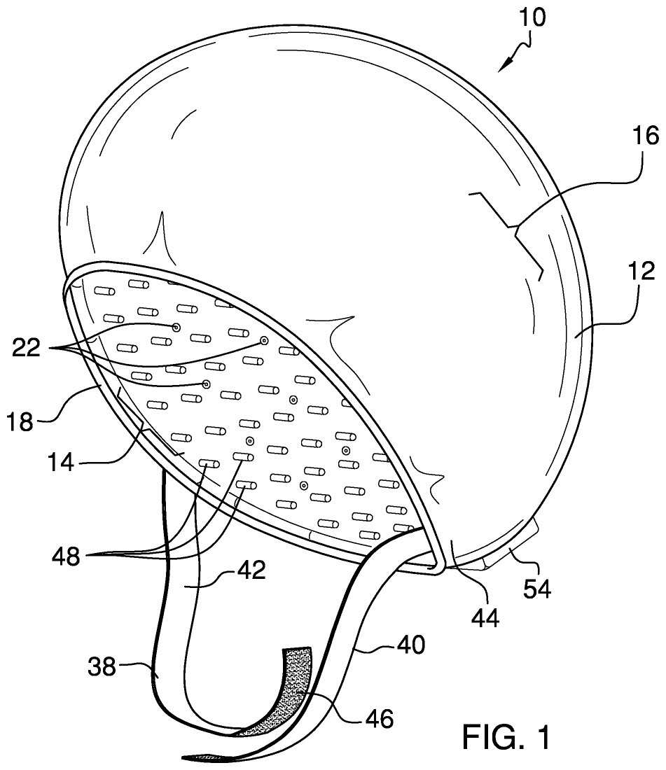

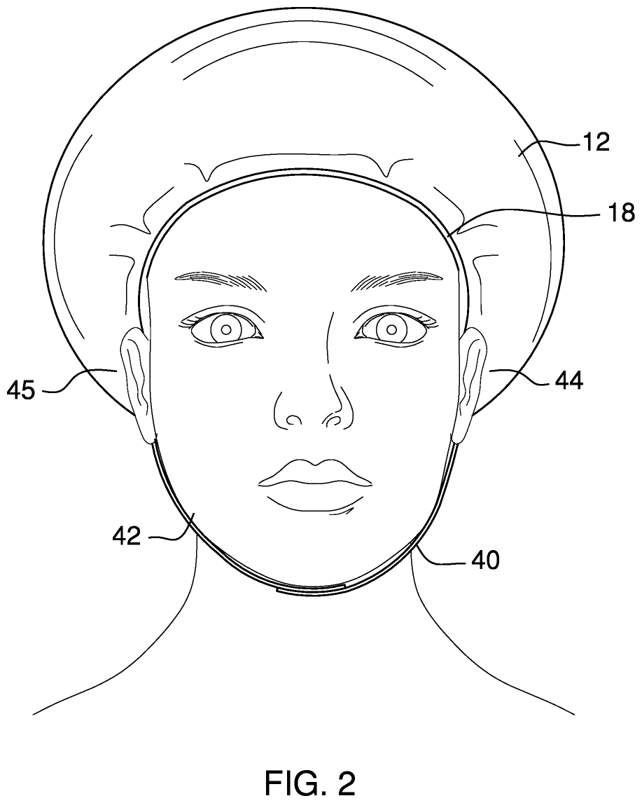

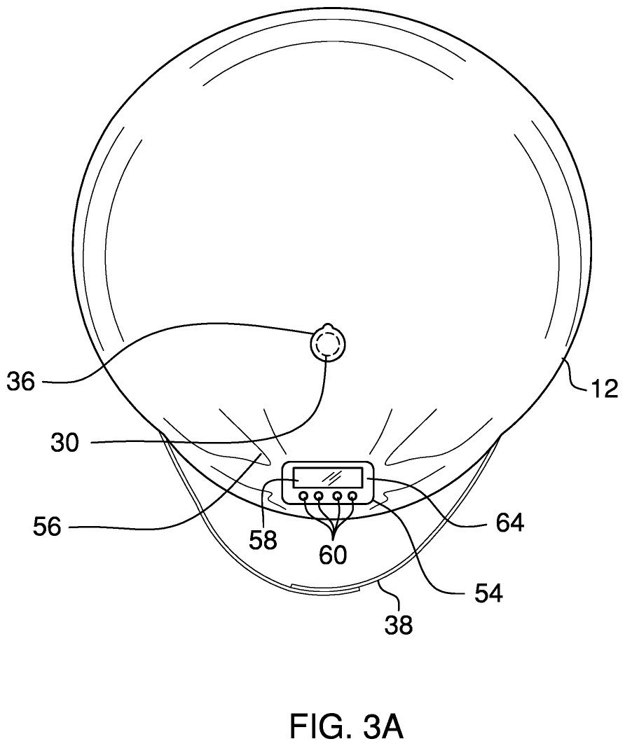

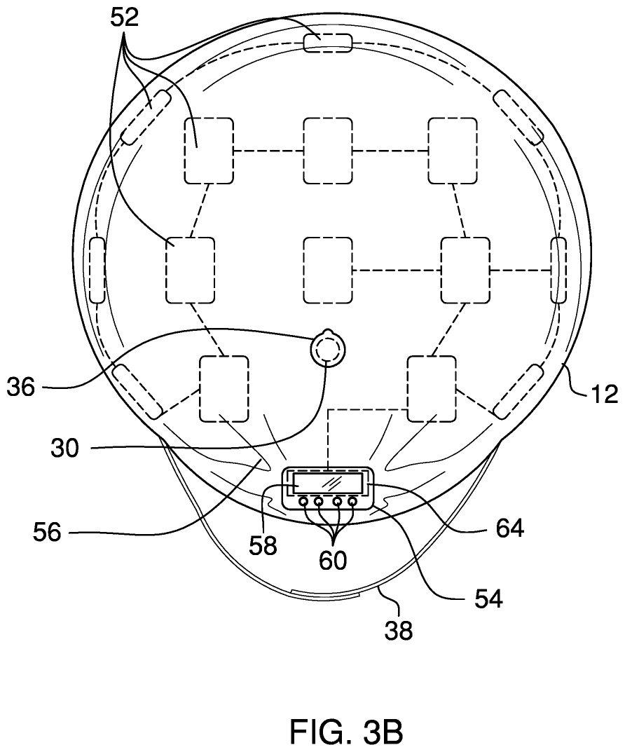

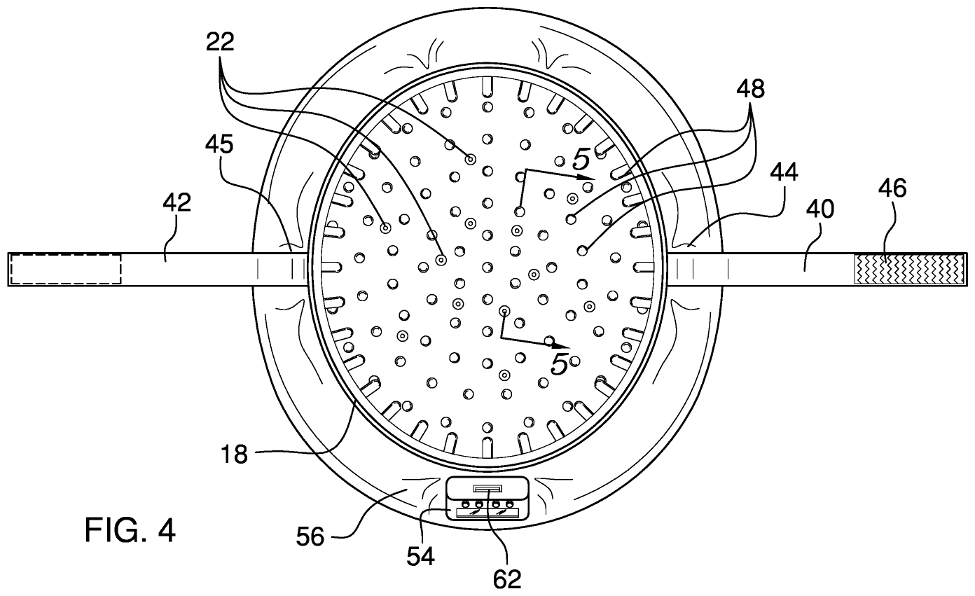

With reference now to the drawings, and in particular to FIGS. 1 through 5 thereof, a new hair treatment device embodying the principles and concepts of an embodiment of the disclosure and generally designated by the reference numeral 10 will be described.

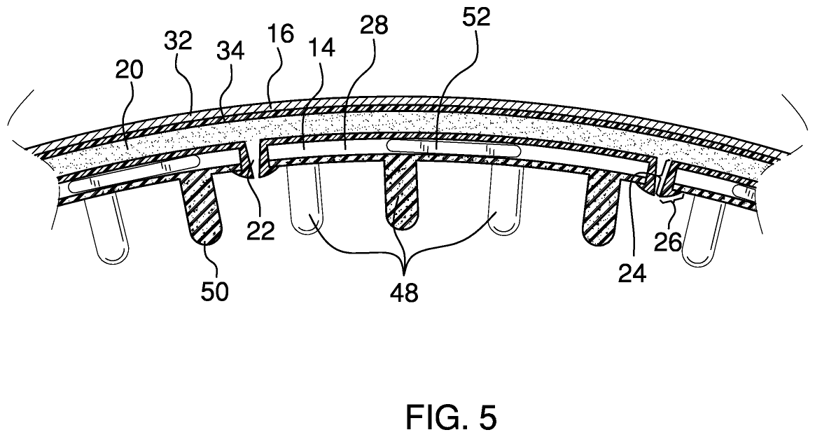

As best illustrated in FIGS. 1 through 5, the hair treatment cap apparatus 10 generally comprises a cap body 12. The cap body 12 has an inner layer 14 and an outer layer 16 joined at a cap edge 18 and defining a cap bladder 20. The inner layer 14 has a plurality of release apertures 22 extending through to the cap bladder 20. Each release aperture 22 may have a tapered shaft portion 24 and a rounded mouth portion 26 extending onto the inner layer 14 (as seen in FIG. 5) to aid the flow of liquid from within the cap bladder 20. The inner layer 14 has a dry cavity 28.

The outer layer 16 of the cap body has a fill aperture 30 extending through to the cap bladder 20. The cap bladder 20 is configured to hold a liquid hair treatment that is filled through the fill aperture 30 and dispersed through the plurality of release apertures 22. The cap body 12 is sufficiently flexible to conform to the shape of a wearer's head. The outer layer 16 may include a soft outer membrane 32 and a waterproof inner membrane 34. The cap edge 18 is elasticized to form a waterproof barrier around the user's head to prevent the liquid hair treatment from entering the user's eyes and to allow the apparatus 10 to be worn while showering. A plug 36 is coupled to the cap body 12 to selectively engage the fill aperture 30.

A strap 38 may be coupled to the cap body 12. The strap 38 includes a strap first half 40 and a strap second half 42 coupled to a cap left portion 44 and a cap right portion 45 of the cap body, respectively. Each of the strap first half 40 and the strap second half 42 has an engagement member 46 to selectively and adjustably engage the strap 38 beneath the wearer's chin. The engagement member 46 may be a selectively engageable fastener such as, but not limited to, hook-and-loop, snaps, buttons, and the like. The strap 38 may be elasticized to provide a snug and comfortable fit.

A plurality of nubs 48 is coupled to the cap body 12. The plurality of nubs 48 is coupled to the inner layer 14. Each of the plurality of nubs 48 may be cylindrical and has a rounded distal end 50. Each of the plurality of nubs 48 may extend perpendicularly from the inner layer 14 and may be a resilient rubberized material for partial elastic deformation while maintaining sufficient rigidity to allow for user comfort while also serving to massage the liquid hair treatment into the wearer's scalp. A plurality of massage motors 52 is coupled to the cap body 12. The plurality of massage motors 52 may be coupled within the dry cavity 28 of the inner layer. The plurality of massage motors 52 causes each of the plurality of nubs 48 to vibrate to assist in the application of the liquid hair treatment and to stimulate blood flow to the user's scalp and hair follicles.

A controller 54 is coupled to the cap body 12. The controller 54 may be coupled to the outer layer 16 of the cap body on a cap rear portion 56 proximal the cap edge 18. The controller 54 may include a display 58 and a plurality of control buttons 60. The controller 54 is in operational communication with the plurality of massage motors 52 to utilize the plurality of control buttons 60 and the display 58 to turn the plurality of massage motors 52 on and off, adjust the strength of vibration, and to create coordinated massage patterns on the user's scalp. The controller 54 may have a charge port 62. A battery 64 is coupled within the controller 54 and is in operational communication with the charge port 62.

In use, the liquid hair treatment is filled into the cap bladder 20 through the fill aperture 30. The plug 36 is then engaged with the fill aperture 30. The user places the cap body 12 onto his or her scalp and engages the strap 38. The controller 54 is then used to manipulate the plurality of massage motors 52 as desired.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A hair treatment cap apparatus comprising:
a cap body, the cap body having an inner layer and an outer layer joined at a cap edge and defining a cap bladder, the inner layer having a plurality of release apertures extending through to the cap bladder, the outer layer having a fill aperture extending through to the cap bladder, the cap bladder being configured to hold a liquid hair treatment that is filled through the fill aperture and dispersed through the plurality of release apertures, the cap body being sufficiently flexible to conform to the shape of a wearer's head, the cap edge being elasticized to form a waterproof barrier around the user's head;
a plug coupled to the cap body, the plug selectively engaging the fill aperture;
a plurality of nubs coupled to the cap body, the plurality of nubs being coupled to the inner layer;
a plurality of massage motors coupled to the cap body, the plurality of massage motors causing each of the plurality of nubs to vibrate;
a controller coupled to the cap body, the controller being in operational communication with the plurality of massage motors; and
a battery coupled within the controller, the battery being in operational communication with the controller.

2. The hair treatment cap apparatus of claim 1 further comprising a strap coupled to the cap body, the strap including a strap first half and a strap second half coupled to a cap left portion and a cap right portion of the cap body, respectively, each of the strap first half and the strap second half having an engagement member to selectively and adjustably engage the strap beneath the wearer's chin.

3. The hair treatment cap apparatus of claim 1 further comprising the inner layer of the cap body having a dry cavity; the plurality of massage motors being coupled within the dry cavity.

4. The hair treatment cap apparatus of claim 1 further comprising the controller including a display and a plurality of control buttons.

5. The hair treatment cap apparatus of claim 1 further comprising the controller being coupled to the outer layer of the cap body on a cap rear portion proximal the cap edge.

6. The hair treatment cap apparatus of claim 1 further comprising the controller having a charge port, the battery being in operational communication with the charge port.

7. The hair treatment cap apparatus of claim 1 further comprising each release aperture having a tapered shaft portion and a rounded mouth portion extending onto the inner layer of the cap body.

8. The hair treatment cap apparatus of claim 1 further comprising each of the plurality of nubs being cylindrical and having a rounded distal end.

9. A hair treatment cap apparatus comprising:
a cap body, the cap body having an inner layer and an outer layer joined at a cap edge and defining a cap bladder, the inner layer having a plurality of release apertures extending through to the cap bladder, each release aperture having a tapered shaft portion and a rounded mouth portion extending onto the inner layer, the inner layer having a dry cavity, the outer layer having a fill aperture extending through to the cap bladder, the cap bladder being configured to hold a liquid hair treatment that is filled through the fill aperture and dispersed through the plurality of release apertures, the cap body being sufficiently flexible to conform to the shape of a wearer's head, the cap edge being elasticized to form a waterproof barrier around the user's head;
a strap coupled to the cap body, the strap including a strap first half and a strap second half coupled to a cap left portion and a cap right portion of the cap body, respectively, each of the strap first half and the strap second half having an engagement member to selectively and adjustably engage the strap beneath the wearer's chin;
a plug coupled to the cap body, the plug selectively engaging the fill aperture;
a plurality of nubs coupled to the cap body, the plurality of nubs being coupled to the inner layer, each of the plurality of nubs being cylindrical and having a rounded distal end;
a plurality of massage motors coupled to the cap body, the plurality of massage motors being coupled within the dry cavity, the plurality of massage motors causing each of the plurality of nubs to vibrate;
a controller coupled to the cap body, the controller being coupled to the outer layer of the cap body on a cap rear portion proximal the cap edge, the controller including a display and a plurality of control buttons, the controller being in operational communication with the plurality of massage motors, the controller having a charge port; and
a battery coupled within the controller, the battery being in operational communication with the charge port of the controller.
